(19) United States
(12) Patent Application Publication
(10) Pub. No.: US 2016/0143911 A1
(43) Pub. Date: May 26, 2016

(54) STABLE AND WATER SOLUBLE PHARMACEUTICAL COMPOSITIONS COMPRISING PEMETREXED

(71) Applicant: SYNTHON B.V., Nijmegen (NL)

(72) Inventors: BOREK ZALUDEK, BLANSKO (CZ); JACOBUS, THEODORUS, HENRICUS EUPEN VAN, NIJMEGEN (NL)

(73) Assignee: SYNTHON B.V., Nijmegen (NL)

(21) Appl. No.: 14/898,429

(22) PCT Filed: Jun. 14, 2013
(86) PCT No.: PCT/EP2013/062412

§ 371 Date: Dec. 14, 2015

(51) Int. Cl. A61K 31/519; A61K 9/16; A61K 9/19

(52) U.S. Cl. CPC A61K 31/519; A61K 9/19; A61K 9/1682

(57) ABSTRACT

The present invention relates to a solid pharmaceutical composition comprising pemetrexed and a solubilizing amount of meglumine or tromethamine. The composition is sufficiently stable for purpose of making pharmaceutical formulations, particularly lyophilized formulations, and is sufficiently soluble in water for purposes of using in parenteral administration.

Pemetrexed is a common name for compound N-[4-[2-(2-amino-4,7-dihydro-4-oxo-1H-pyrrolo [2,3-d]pyrimidin-5-yl)ethyl]benzoyl]-L-glutamic acid of formula (1)

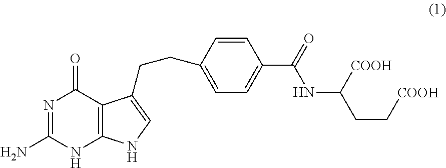 [SMILES: Nc1nc(=O)c2c(CCc3ccc(C(=O)NC(CCC(=O)O)C(=O)O)cc3)c[nH]c2[nH]1]

The compound has been first disclosed in EP 432677.

Pemetrexed is a pharmaceutically active compound, which is used, e.g., in treatment of malignant pleural mesothelioma and non-small cell lung cancer.

As pemetrexed of the above formula (1) is a bivalent acid (a diacid) comprising two carboxylic groups, it may form various types of salts with bases. On the other hand, pemetrexed has basic nitrogens and, accordingly, it may form acid addition salts with various acids. The commercially available product, sold, e.g., under the brand name ALIMTA by Eli Lilly, comprises hydrated disodium salt of pemetrexed as the active substance and is supplied as a sterile lyophilized powder for intravenous infusion available in single-dose vials. The preparation of lyophilized pemetrexed disodium composition is disclosed in U.S. Pat. No. 7,138,521.

U.S. Pat. No. 7,138,521 also describes a stable crystalline heptahydrate form of pemetrexed disodium having a characteristic X-ray diffraction pattern. The patent states that pemetrexed disodium can exist in the form of a heptahydrate which is much more stable than the previously known 2.5 hydrate and shows that the primary advantage of the heptahydrate crystalline form over the 2.5 hydrate crystal form is its stability and also with respect to formation of related substances. It also shows that when the heptahydrate is subjected to elevated temperatures, low humidity, and/or vacuum, it converts to the 2.5 hydrate crystal form by loss of water.

The above patent shows that problems may arise because of conversions between different polymorphic forms of pemetrexed disodium when exposed to elevated temperatures, low humidity, etc. Formulation processes may involve a variety of the above mentioned adverse conditions, resulting in a possibility that the stability of the final product may be affected.

The main reason why pemetrexed has not been used in pharmaceutical compositions in its free diacid form (Formula (1)) so far, is apparently the fact that pemetrexed diacid is only sparingly soluble in water. Therefore, it cannot be used in water soluble compositions formulated for parenteral applications.

Pemetrexed diacid of the above formula (1) has indeed a potential for use in medicine as it is a well defined stable compound, which may be produced by a standard process and purified to the desired level of purity. Thus, for instance, formation and isolation of pemetrexed diacid from a mixture of water and ethanol having a pH of 2.5-3.5 is disclosed in

U.S. Pat. No. 7,138,521. Formation and isolation of pemetrexed diacid from an aqueous solution having a pH of 5 is disclosed in U.S. patent No. 5,416,211. Formation and isolation of pemetrexed diacid from an aqueous solution having a pH of 2.8-3.1 is disclosed in U.S. Pat. No. 6,262,262.

Various crystalline forms of pemetrexed diacid as well as methods for obtaining them have been disclosed in US 8,088,919, EP 2351755 and EP 2129674.

Highly pure pemetrexed diacid may be obtained according to a process disclosed in WO2008/021410A2.

In essence, it is desirable to formulate the pemetrexed diacid in a stable and water soluble pharmaceutical composition.

### BRIEF DESCRIPTION OF THE INVENTION

The present invention relates to a novel solid pharmaceutical composition comprising pemetrexed. The composition is sufficiently stable for purpose of making pharmaceutical formulations, particularly lyophilized formulations, and is sufficiently soluble in water for purposes of using in parenteral administration.

In the first aspect, the invention relates to a solid pharmaceutical composition comprising pemetrexed of formula (1)

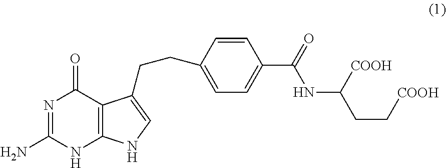 [SMILES: Nc1nc(=O)c2c(CCc3ccc(C(=O)NC(CCC(=O)O)C(=O)O)cc3)c[nH]c2[nH]1]

and a solubilizing amount of meglumine of formula (2),

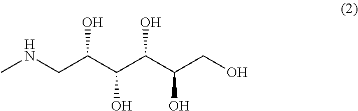

or tromethamine of formula (3),

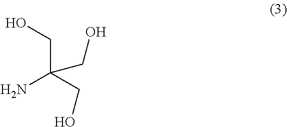

wherein the solubilizing amount of meglumine is from 80 to 110 weight per cent in respect to pemetrexed, and wherein the solubilizing amount of tromethamine is from 50 to 75 weight per cent in respect to pemetrexed.

In a particular aspect, the composition further comprises at least one pharmaceutically acceptable excipient, advantageously a sugar alcohol, more advantageously mannitol.

In a particular aspect, the composition is an amorphous form.

In a particular aspect, the pharmaceutical composition is a lyophilized powder useful for reconstitution into an infusion solution by an infusion liquid.

In a second aspect, the invention relates to a process for making a solid and water soluble pharmaceutical composition comprising pemetrexed, said process comprising a step of combining, upon formation a solution, the diacid form of pemetrexed of formula (1) with solubilizing amount of meglumine of formula (2) or tromethamine of formula (3) in a solvent comprising water, followed by a step of removal the solvent. In an embodiment, at least one pharmaceutically acceptable excipient is also added to said solution. In an embodiment, the solvent is removed by lyophilization.

In a particular aspect, the solubilizing amount of meglumine is from 80 to 110 weight per cent in respect to pemetrexed and the solubilizing amount of tromethamine is from 50 to 75 weight per cent in respect to pemetrexed.

### DETAILED DESCRIPTION OF THE INVENTION

The present invention relates to a solid water soluble pharmaceutical composition comprising pemetrexed of formula (1). In making the composition, the pemetrexed is used in its diacid form, i.e. the invention does not employ any pemetrexed salt as the starting material. The fact that pemetrexed needs not to be converted into a salt prior to using it in making the pharmaceutical composition is advantageous because pemetrexed is relatively unstable compound and any way of reducing the number of process steps involved in the manufacture of the starting product reduces the potential for degradation. Pemetrexed diacid is even more stable than the disodium salt during storage, thus it is advantageous starting material from technological aspects.

Throughout the disclosure and claims, the words “pemetrexed” and “pemetrexed diacid” are used in equivalent meaning, unless specifically stated otherwise. Both correspond with the formula (1).

As stated above, pemetrexed is inherently only sparingly soluble in water. It was now found with surprise that a solid and water soluble pharmaceutical composition with good stability may be made by combining pemetrexed diacid with solubilizing amount of meglumine or tromethamine. Meglumine, ((2R,3R,4R,5S)-6-(Methylamino)hexane-1,2,3,4,5-pentol of formula (2), is an aminoalcohol derived from sorbitol. Tromethamine, 2-amino-2-hydroxymethyl-propane-1,3-diol of formula (3), is also an aminoalcohol, which is extensively used in biochemistry. Both compounds are known, commercially available and pharmaceutically acceptable. Because of low toxicity and other favourable properties, it has been assumed that they can suitable even for parenteral applications. Thus, the solid and water soluble composition according to present invention comprises pemetrexed and a solubilizing amount of meglumine or tromethamine. The “solubilizing amount of meglumine” is typically from 80 to 110 weight per cent in respect to pemetrexed (0.8 to 1.1 g per 1 g of pemetrexed (diacid)), advantageously between 90 and 105 weight per cent in respect to pemetrexed (0.9 to 1.05 g per 1 g of pemetrexed (diacid)). The “solubilizing amount of tromethamine” is typically from 50 to 75 weight per cent in respect to pemetrexed (0.5 to 0.75 g per 1 g of pemetrexed (diacid)), advantageously between 55 and 70 weight per cent in respect to pemetrexed (0.55 to 0.7 g per 1 g of pemetrexed (diacid)).

The composition may be advantageously formulated into dosage forms for parenteral administration. For said purposes, it is advantageous that the composition is in amorphous form. In a particular aspect, the pharmaceutical composition is formulated as a lyophilized powder useful for reconstitution into an infusion solution by an infusion liquid.

The composition of the present invention further comprises at least one pharmaceutically acceptable excipient. The pharmaceutically acceptable excipients for purpose of the present invention, particularly those useful for making the lyophilized pharmaceutical formulations, are preferably water soluble and may include one or more of: diluents or bulking agents including one or more sugars such as dextrose, sucrose, mannose, lactose, trehalose and the like, one or more sugar alcohols such as mannitol, xylitol and the like; antibacterial preservatives, including one or more of thiomersal, benzalkonium chloride, benzethonium chloride; pH-adjustors such as hydrochloric acid or sodium hydroxide; buffers including one or more of acetate, citrate, tartrate, phosphate, benzoate, and bicarbonate buffers; chelating agents such as disodium edetate; antioxidants including ascorbic acid, glutathione, L-cysteine, lipoic acid and the like; tonicity contributors including one or more of sodium chloride, potassium chloride, dextrose, mannitol, and lactose. The addition of a sugar or sugar alcohol can improve the stability of pemetrexed formulations. Thus, a suitable lyophilized pharmaceutical formulation according to various aspects of the present invention may preferably also comprise at least one sugar or sugar alcohol, advantageously mannitol.

In various advantageous embodiments, the sugar or sugar alcohol is present in amounts from 0,1 g to 2 g per 1 g of pemetrexed.

The solid product may comprise residual water and/or organic solvent. Typically, the product comprises less than 10% of these volatile components.

The unit dose of the pharmaceutical composition according to the present invention typically comprises from 50 to 1500 mg of pemetrexed, calculated as the diacid form. Advantageously, the unit dose comprises 100 mg, 250 mg, 500 mg or 1000 mg of pemetrexed. Thus, in a specific aspect, the invention includes a vial or similar container comprising a dose amount of the composition of the invention. Any suitable sterile vial or container fit for the sterile storage of a pharmaceutical such as pemetrexed for extended periods of time may be used. Suitable containers can be glass vials, polypropylene or polyethylene vials or other special purpose containers.

A further aspect of the invention includes a kit and / or pharmaceutical container for holding the pemetrexed-containing compositions described herein. The kit contains at least one pharmaceutically acceptable vial or container containing one or more doses of the pemetrexed-containing formulations/compositions as well as other pharmaceutically necessary materials for storing and/or administering the drug, including instructions for storage and use, infusion bag or container with an infusion diluent etc.

The solid and water soluble pharmaceutical composition of pemetrexed according to present invention can be made by a process comprising a step of combining, upon formation a solution, the diacid form of pemetrexed of formula (1) with the solubilizing amount of meglumine of formula (2) or tromethamine of formula (3) in a solvent comprising water, followed by a step of removal the solvent. Said solvent may be water per se or it may comprise a mixture of water with a pharmaceutically acceptable cosolvent, particularly that which is susceptible to lyophilization. Such cosolvents are known in the art, an example is tert. butanol. Water must be of pharmaceutically acceptable quality. Typically, water in quality “for injections”, as defined in acknowledged Pharmacopoeias, is used.

The final concentration of pemetrexed in the solution is not particularly limited and is rather directed by technological aspects, which comprise the need of final removal of the solvent. Thus, in practice, a suitable but not limiting concentration of pemetrexed in the solution is from 10 and 40 mg/ml, preferably between 15 and 30 mg/ml. The “solubilizing amount” of meglumine or tromethamine is in certain aspect dependent on the desired final concentration. From the same technological reasons, it is advantageous that the solubilizing amount of meglumine is from 80 to 110 weight per cent, advantageously between 90 and 105 weight per cent, in respect to pemetrexed, and the solubilizing amount of tromethamine is from 50 to 75 weight per cent, advantageously between 55 and 70 weight per cent, in respect to pemetrexed.

The process typically comprises weighing the respective ingredients (pemetrexed acid and the solubilizer) and dissolving them in water solvent, preferably under stirring. Advantageously, water is first deoxygenated by a suitable technique, e.g. by saturating it by an inert gas, by deaerating with ultrasound etc.

The dissolution process is preferably conducted in the atmosphere of an inert gas such as nitrogen or argon.

The dissolution is typically carried out at ambient temperature.

In practice, it is advantageous to prepare a solution of meglumine or tromethamine in the water solvent and to add pemetrexed into such solution.

In further, at least one pharmaceutically acceptable excipient may be added to said solution. The nonexhaustive list of such excipients was given above. The excipients must be sufficiently soluble in the solvent system. As stated above, typical excipient is a sugar or a sugar alcohol, advantageously mannitol, which may be, in some embodiments, present in amounts from 0,1 g to 2 g per 1 g of pemetrexed.

In the final stage, pH of the solution is optionally adjusted to the desired value, which is typically from 7.0 to 9.0, preferably from 7.5 to 8.5. The pH adjustor may be any suitable pharmaceutically acceptable acid, base, salt or a combination thereof.

In certain embodiments, the obtained solution is filtered and sterilized and filled into vials comprising the desired amount of pemetrexed per vial.

In the next step, the solvent is removed from the composition. Typically, water is removed by lyophilization (freeze-drying) under suitable conditions. Freeze drying can be conducted at temperatures from about −10 to about −50° C., under vacuum in the range of about 0.5 to about 50 Pa.

In an advantageous embodiment, the subject of the lyophilization process is the content of vials prepared as shown above. As a result, the lyophilization process yields a solid pharmaceutical composition comprising a unit dose of pemetrexed, which typically comprises from 50 to 1500 mg of pemetrexed, calculated as the diacid form, advantageously 100mg, 250 mg, 500 mg or 1000 mg of pemetrexed.

In the last step of the overall process, the vials are closed by a suitable stopper, labeled and packed into suitable container.

The compositions and formulations of the present invention are particularly suited for parenteral administration. For such administration, the composition is reconstituted prior to its use by an appropriate liquid medium. The medium may include sterile water, a pH buffered solution, sodium chloride solution or a dextrose solution.

The compositions of the present invention may be used in medicine, particularly for treating a pemetrexed sensitive disease in mammals. Thus, in yet another aspect of the invention, there are provided methods of treating a pemetrexed sensitive disease in mammals. Pemetrexed sensitive diseases include, but are not limited to, cancers, such as malignant pleural mesothelioma and non-small cell lung cancer. The use or methods include administering an effective amount of a pemetrexed-containing composition as described herein to a mammal in need thereof.

The following examples illustrate the invention.

### EXAMPLES

### Example 1

Solid pharmaceutical composition comprising pemetrexed and meglumine (50 mg pemetrexed per unit)

Under inert atmosphere of nitrogen, pemetrexed diacid (750 mg) was dissolved in solution of meglumine (2.2 molar equivalents related to active substance: 754.0 mg) in 25 ml of Water for injection. The clear, yellowish solution was easily formed. After that mannitol (750 mg) was added and dissolved under stirring. The pH of the solution was adjusted to about 7.5 by 1 M hydrochloric acid and the weight of the solution was adjusted to 30 grams by Water for injection. The solution was filtered through PVDF filter 0.2 microns, filed in clear 10 R vials per 2 grams and freeze dried using the program presented in the table.

| | Duration | Temperature | Vacuum | Safety Pressure |
|---|---|---|---|---|
| Process Phase | (hh:mm) | (° C.) | (mbar) | (mbar) |
| Loading | 00:00 | +5 | | |
| Fast freezing | 01:00 | −45 | | |
| Freezing | 01:30 | −45 | | |
| Freezing | 00:45 | −15 | | |
| Annealing | 01:00 | −15 | | |
| Freezing | 01:30 | −45 | | |
| Freezing | 02:00 | −45 | | |
| Evacuation 1 | 00:10 | −45 | 0.37 | 0.63 |
| Sublimation | 06:35 | +10 | 0.37 | 0.63 |
| Sublimation | 05:50 | +10 | 0.37 | 0.63 |
| Evacuation 2 | 00:10 | +10 | 0.06 | 0.63 |
| Second. Drying | 03:20 | +30 | 0.06 | 0.63 |
| Second. Drying | 05:00 | +30 | 0.06 | 0.63 |

Stability

A stability study has been performed in order to compare the composition of the present example with the commercial product ALIMTA 100 mg and 500 mg. Impurities present in the pemetrexed-comprising formulations during stability studies performed were detected by high performance liquid chromatography (HPLC) equipped with UV detector at a suitable wavelength (typically 227 nm) and calculated on a normalized peak area response (“PAR”) basis. As an acceptable limit, demonstrating sufficient stability at the corresponding sampling point, the sum of peaks of all individual degradants in the inventive compositions should not exceed 2% of the total PAR. The peak size of any individual degradant should not exceed 1% of the total PAR. In the tables below, the “UN” denotes an unknown impurity, Impurities A, B, C and D have known structure (Ph.Eur.).

The results are presented in tables:

Alimta 500 mg, Batch A721520E

| | | | | 3 months | 6 months |
|---|---|---|---|---|---|
| | | | 2 weeks | 40° C./ | 40° C./ |
| Name | RRT | Zero | 50° C. | 75% RH | 75% RH |
| UN | 0.33 | nd | nd | nd | 0.01 |
| UN | 0.37 | 0.04 | 0.04 | 0.05 | 0.04 |
| UN | 0.39 | nd | nd | 0.01 | nd |
| UN | 0.46 | 0.15 | 0.14 | 0.13 | 0.14 |
| Impurity A | 0.58 | 0.04 | 0.04 | 0.04 | 0.04 |
| Impurity B and C | 0.66 | 0.11 | 0.11 | 0.08 | 0.09 |
| UN | 0.74 | 0.01 | 0.01 | 0.01 | 0.01 |
| UN | 0.88 | 0.01 | nd | 0.01 | 0.02 |
| UN | 0.91 | 0.01 | 0.01 | 0.01 | 0.01 |
| UN | 1.78 | nd | 0.01 | 0.01 | 0.01 |
| ΣIMP (% IN) | | 0.37 | 0.36 | 0.35 | 0.37 |

Alimta 100 mg, Batch A693326

| | | | | 1 month | 6 months |
|---|---|---|---|---|---|
| | | | 2 weeks | 40° C./ | 40° C./ |
| Name | RRT | Zero | 50° C. | 75% RH | 75% RH |
| UN | 0.34 | 0.02 | nd | nd | nd |
| UN | 0.41 | 0.01 | 0.01 | 0.01 | nd |
| UN | 0.48 | 0.06 | 0.05 | 0.01 | nd |
| UN | 0.50 | nd | nd | 0.02 | 0.05 |
| Impurity A | 0.64 | 0.03 | 0.03 | 0.03 | 0.03 |
| Impurity B and C | 0.72 | 0.04 | 0.04 | 0.03 | 0.03 |
| UN | 0.81 | 0.02 | 0.02 | 0.02 | 0.02 |
| UN | 0.94 | 0.01 | nd | nd | nd |
| UN | 0.96 | 0.01 | 0.01 | nd | 0.01 |
| UN | 0.99 | 0.02 | 0.01 | 0.01 | 0.01 |
| UN | 2.01 | nd | nd | nd | 0.01 |
| ΣIMP (% IN) | | 0.22 | 0.17 | 0.13 | 0.16 |

### Composition of Example 1

| | | | | 1 month |
|---|---|---|---|---|
| | | | 2 weeks | 40° C./ |
| Name | RRT | ZERO | 50° C. | 75% RH |
| Pemetrexed (% in) | 1.00 | 99.74 | 99.65 | 99.73 |
| imp_A | 0.78 | 0.04 | 0.05 | 0.04 |
| imp_D | 0.91 | <0.04% | <0.04% | <0.04% |
| UN | 0.92 | 0.12 | 0.11 | 0.12 |
| imp_B | 0.96 | <0.04% | <0.04% | <0.04% |
| imp_C | 0.98 | <0.04% | <0.04% | <0.04% |
| ΣIMP | | 0.16 | 0.16 | 0.16 |
| (≧0.04% IN) | | | | |
| ΣIMP | | 0.10 | 0.19 | 0.11 |
| (<0.04% IN) | | | | |
| ΣIMP (% IN) | | 0.26 | 0.35 | 0.27 |

It can be concluded that the lyophilized composition of the Example 1 is fully comparable to commercial pemetrexed disodium composition from stability point of view.

### Example 2

Solid pharmaceutical composition comprising pemetrexed and tromethamine (50 mg pemetrexed per unit)

Under inert atmosphere of nitrogen, pemetrexed diacid (750 mg) was dissolved in solution of tromethamine (2.1 molar equivalents related to active substance: 446.4 mg) in 25 grams of Water for injection. After that, mannitol (750 mg) was added and dissolved under stirring. The pH of the solution was adjusted to about 7.5 by 1 M hydrochloric acid and the weight of the solution was adjusted to 30 grams by Water for injection. The solution was filtered through PVDF filter 0.2 microns, filed in clear 10 R vials per 2 grams and freeze dried using the program presented in the table.

| | Duration | Temperature | Vacuum | Safety Pressure |
|---|---|---|---|---|
| Process Phase | (hh:mm) | (° C.) | (mbar) | (mbar) |
| Loading | 00:00 | +5 | | |
| Fast freezing | 01:00 | −45 | | |
| Freezing | 01:30 | −45 | | |
| Freezing | 00:45 | −15 | | |
| Annealing | 01:00 | −15 | | |
| Freezing | 01:30 | −45 | | |
| Freezing | 02:00 | −45 | | |
| Evacuation 1 | 00:10 | −45 | 0.37 | 0.63 |
| Sublimation | 06:35 | +10 | 0.37 | 0.63 |
| Sublimation | 05:50 | +10 | 0.37 | 0.63 |
| Evacuation 2 | 00:10 | +10 | 0.06 | 0.63 |
| Second. Drying | 03:20 | +30 | 0.06 | 0.63 |
| Second. Drying | 05:00 | +30 | 0.06 | 0.63 |

1. A solid pharmaceutical composition comprising pemetrexed of formula (1) 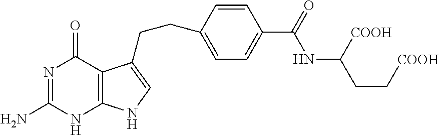 [SMILES: Nc1nc(=O)c2c(CCc3ccc(C(=O)NC(CCC(=O)O)C(=O)O)cc3)c[nH]c2[nH]1]
and a solubilizing amount of meglumine of formula (2), 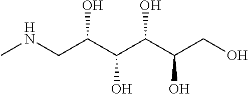
or tromethamine of formula (3) 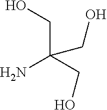
wherein the solubilizing amount of megiumine is from 80 to 110 weight per cent in respect to pemetrexed, and wherein the solubilizing amount of tromethamine is from 50 to 75 weight per cent in respect to pemetrexed.

2. The composition according to claim 1 further comprising at least one pharmaceutically acceptable excipient.

3. The composition according to claim 2, wherein the excipient is a sugar alcohol, advantageously mannitol.

4. The composition according to claim 1 in amorphous form.

5. The composition according to claim 1, which is a lyophilized powder useful for reconstitution into an infusion solution by an infusion liquid.

6. The composition according to claim 5 comprising a single dose of pemetrexed.

7. A process for making the pharmaceutical composition according to claim 1, said process comprising a step of combining, upon formation a solution, the diacid form of pemetrexed of formula (1) with solubilizing amount of meglumine of formula (2) or tromethamine of formula (3) in a solvent comprising water.

8. The process according to claim 7 further comprising adding at least one pharmaceutically acceptable excipient to said solution.

9. The process according to claim 7, further comprising adjustment of pH to a value from 7.0 to 9.0, preferably from 7.5 to 8.5.

10. The process according to claim 7, further comprising a next step of removal the solvent, advantageously by lyophilization.

11. The process according to claim 7, wherein the solubilizing amount of meglumine is from 80 to 110 weight per cent in respect to pemetrexed or the solubilizing amount of tromethamine is from 50 to 75 weight per cent in respect to pemetrexed.
